(19) United States
(12) Patent Application Publication
(10) Pub. No.: US 2012/0309069 A1
(43) Pub. Date: Dec. 6, 2012

(54) Yeast for Fermentation

(76) Inventors: Philip John Livingstone Bell, Turramurra (AU); Paul Victor Attfield, Mount Colah (AU)

(21) Appl. No.: 13/498,548

(22) PCT Filed: Sep. 27, 2010
(86) PCT No.: PCT/AU10/01262

§ 371 Date: Aug. 22, 2012

(30) Foreign Application Priority Data
Sep. 28, 2009 (AU) 2009904730

(51) Int. Cl. C12P 7/06; C09K 3/00; C07C 31/08; C12N 1/16; C12P 7/10

(52) U.S. Cl. 435/165; 435/255.2; 435/161; 568/840; 252/182.12

(57) ABSTRACT

The invention relates to an isolated Saccharomyces cerevisiae strain having NMI accession no. V09/024011, and to a method of producing ethanol and distiller's grains using the strain.

### FIELD

The present invention relates to a Saccharomyces yeast strain having improved ability to ferment sugars to ethanol, and to the use of such a Saccharomyces yeast strain in the production of ethanol.

### BACKGROUND

The production of ethanol as a bio-fuel has become a major industry, with in excess of 15 billion gallons of ethanol being produced world wide in 2008.

Yeast which are used for production of ethanol for use as fuel, such as in the corn ethanol industry, require several characteristics to ensure cost effective production of the ethanol. These characteristics include ethanol tolerance, low by-product yield, rapid fermentation, temperature tolerance, and the ability to limit the amount of residual sugars remaining in the ferment. Such characteristics have a marked effect on the viability of the industrial ethanol production process.

Yeast of the genus Saccharomyces exhibit many of the characteristics required for production of ethanol in the fuel industry. In particular, strains of Saccharomyces cerevisiae are widely used for the production of ethanol in the fuel ethanol industry. Strains of Saccharomyces cerevisiae that are widely used in the fuel ethanol industry have the ability to produce high yields of ethanol under fermentation conditions found in, for example, the fermentation of corn mash. Examples of such strains include strains sold under the names Fali, Thermosacc Dry, Ethanol Red and Danstil BG-1, which are used in commercially available ethanol yeast products.

Strains of Saccharomyces cerevisiae are used in the fuel ethanol industry to ferment sugars such as glucose, fructose, sucrose and maltose to produce ethanol via the glycolytic pathway. These sugars are obtained from sources such as corn and other grains, sugar juice, molasses, grape juice, fruit juices, and starchy root vegetables.

Although strains of Saccharomyces cerevisiae currently used in the fuel ethanol industry are well suited to ethanol production, there is an increasing need for improvements in the efficiency of ethanol production owing to the increased demand for ethanol as a fuel.

There is therefore a need for new strains of Saccharomyces cerevisiae capable of improving the efficiency of ethanol production.

### SUMMARY

A first aspect provides an isolated Saccharomyces cerevisiae strain having NMI accession no. V09/024,011.

A second aspect provides a method of producing ethanol, comprising incubating a Saccharomyces cerevisiae strain having NMI accession no. V09/024,011 with a substrate comprising fermentable sugars under conditions which allow fermentation of the fermentable sugars to produce ethanol.

A third aspect provides a method of producing ethanol, comprising:
(a) incubating a Saccharomyces cerevisiae strain having NMI accession no. V09/024,011 with a substrate comprising fermentable sugars under conditions which allow fermentation of the fermentable sugars to produce ethanol; and
(b) isolating the ethanol.

A fourth aspect provides ethanol produced by the method of the second or third aspect.

A fifth aspect provides use of a Saccharomyces cerevisiae strain having NMI accession no. V09/024,011 in the production of ethanol.

A sixth aspect provides a method of producing distiller's residue comprising incubating a Saccharomyces cerevisiae strain having NMI accession no. V09/024,011 with a substrate comprising fermentable sugars under conditions which allow fermentation of the fermentable sugar to produce ethanol and distiller's residue.

A seventh aspect provides a method of producing distiller's residue comprising:
(a) incubating a Saccharomyces cerevisiae strain having NMI accession no. V09/024,011 with a substrate comprising fermentable sugars under conditions which allow fermentation of the fermentable sugar to produce ethanol and distiller's grains or equivalent residue;
(b) isolating the distiller's residue.

An eighth aspect provides distiller's residue produced by the method of the sixth or seventh aspect.

A ninth aspect provides use of a Saccharomyces cerevisiae strain having NMI accession no. V09/024,011 in the production of distiller's residue.

A tenth aspect provides a method of producing ethanol comprising:
(a) incubating a Saccharomyces cerevisiae strain having NMI accession no. V09/024,011 with a substrate comprising fermentable sugars under conditions which allow fermentation of the fermentable sugars to produce ethanol; and
(b) recovering Saccharomyces cerevisiae strain V09/024,011 following fermentation of at least a portion of the fermentable sugars and repeating step (a), and optionally step (b), with the recovered Saccharomyces cerevisiae strain V09/024,011.

An eleventh aspect provides a composition comprising a Saccharomyces cerevisiae Strain having NMI accession no. V09/024,011.

### BRIEF DESCRIPTION OF THE FIGURE

FIG. 1 shows the results of gel electrophoresis following PCR amplification of yeast strains using primer Ty1R1 (A) or Ty3R1 (B). The lanes for A and B are as follows: Lanes 1 and 7: Size marker (Roche DNA molecular, weight marker X (0.07-12.2 Kbp); Lane 2: Ethanol Red; Lane 3: Fali; Lane 4: Thermosacc Dry; Lane 5: strain V09/024,011; Lane 6: BG-1.

### DETAILED DESCRIPTION

The invention relates to a strain of Saccharomyces cerevisiae deposited under the Budapest Treaty at the National Measurement Institute (NMI) having deposit accession no. V09/024,011 (also referred to herein as “strain V09/024,011”).

The inventors have found that strain V09/024,011 has a number of properties which make it well suited to the industrial production of ethanol. The inventors have compared the ability of strain V09/024,011 to ferment substrate comprising fermentable sugars at temperatures as high as 34 deg Celsius with that of the commercially available fuel ethanol yeast strains Fali, Thermosacc Dry and Ethanol Red. The inventors have found that strain V09/024,011 produces greater ethanol concentrations, and less unwanted by-products, per gram of ethanol produced than the commercially available fuel ethanol strains Fali, Thermosacc Dry and Ethanol Red.

The inventors have found that strain V09/024,011 has the following defining properties:
(a) the ability to produce higher ethanol concentrations than Saccharomyces cerevisiae strains Fali, Thermosacc Dry and Ethanol Red under the fermentation conditions set out in Examples 3, 4 and 5;
(b) the ability to produce lower levels of by-products, such as glycerol and acetate, than Saccharomyces cerevisiae strains Fali, Thermosacc Dry and Ethanol Red under the fermentation conditions set out in Examples 3, 4 and 5; and
(c) the ability to reduce the amount of residual glucose following fermentation to a greater extent than Saccharomyces cerevisiae strains Fali, Thermosacc Dry and Ethanol Red under the fermentation conditions set out in Examples 3, 4 and 5.

The inventors have found that strain V09/024011 is able to efficiently ferment substrate comprising fermentable sugars at 34 deg Celsius to produce ethanol. As described herein, strain V09/024011 produces higher concentrations of ethanol, and less glycerol and acetate, from fermentation of the fermentable sugars in corn mash at 34 deg Celsius than Saccharomyces cerevisiae strains used in the fuel ethanol industry prior to the present invention, such as strains Fali, Thermosacc Dry and Ethanol Red. The inventors have found that strains of Saccharomyces cerevisiae used in the fuel ethanol industry prior to the present invention do not ferment as efficiently as strain V09/024,011 in corn mash at 34 deg Celsius. The ability to ferment substrate comprising fermentable sugars efficiently at 34 deg Celsius permits ethanol production to be carried out efficiently. For example, the ability to efficiently ferment starch-based substrates at 34 deg Celsius means that: less water is required in order to cool the fermentation to a temperature at which the yeast can operate efficiently; hydrolysis of starch in the fermentation is more efficient because enzymes for hydrolysis of starch are more active at higher temperatures; and less energy is required to heat the fermentation to distillation temperature once the fermentation is complete.

In addition, strain V09/024,011 is capable of utilizing xylose as a sole carbon source for growth. In this regard, strain V09/024,011 is capable of a three-fold to 10-fold increase in biomass in Test T1. As a consequence, strain V09/024,011 can be readily distinguished from:
(a) naturally occurring strains of Saccharomyces;
(b) contaminating strains of Saccharomyces; and
(c) other strains used in the ethanol industry that do not have the ethanol producing capabilities of strain V09/024,011 and/or do not exhibit a greater than 3-fold increase in biomass in Test T1.

The ability of strain V09/024,011 to grow on xylose as a sole carbon source more rapidly than naturally occurring strains of Saccharomyces means that strain V09/024,011 can be readily isolated from mixed populations of other Saccharomyces by simply plating the population on medium containing xylose as a sole carbon source. As naturally occurring strains of Saccharomyces are not capable of growth on xylose at the rate at which strain V09/024,011 grows on xylose, strain V09/024,011 is readily differentiated from naturally occurring strains of Saccharomyces and strains of Saccharomyces used in the ethanol industry prior to the present invention such as Fali, Thermosacc Dry and Ethanol Red.

The invention also relates to methods for the production of ethanol using strain V09/024,011. In one form, strain V09/024,011 is incubated with a substrate comprising fermentable sugars under conditions that allow fermentation of the fermentable sugars. As used herein, a “substrate” is a medium suitable for supporting fermentation by Saccharomyces. The fermentable sugars may be one or more of glucose, galactose, maltose, fructose and sucrose. Typically, the fermentable sugar is glucose. The source of the fermentable sugar in the substrate may be any source which contains fermentable sugar. The fermentable sugar in the substrate may be, for example, from any one or more of the following sources: hydrolysed starch, hydrolysed cellulose, molasses (from sugar cane or sugar beet), sugar cane juice, sugar beet juice, grape juice, fruit juice, glucose, hydrolysed maltodextrins, raw sugar juice, galactose, sucrose, any other forms of fermentable sugars, or mixtures thereof. In one form, the source of fermentable sugar in the substrate is hydrolysed starch. Starch may be obtained from any starch rich crops. Examples of starch rich crops include corn, wheat, barley, cassava, sorghum, sweet potato, millet, rice, or any other starch rich crops. In preparing the substrate, the crop is typically crushed and mixed with water and hydrolytic enzyme(s) under conditions which result in hydrolysis of the starch and release of fermentable sugars such as glucose. Typical enzymes for hydrolysis of the starch include α-amylase, amyloglucosidase, pullulanase, β-amylase, glucoamylase, or mixtures thereof. Enzymes suitable for hydrolysis are available from, for example, Novozymes or Genencor Inc. In a particular form, substrate is provided in the form of corn mash. Methods for preparation of corn mash are known in the art and described in, for example, Thomas, K. C. et al., (2001) Journal of Applied Microbiology, volume 90, pages 819-828. Methods for the preparation of starch-based substrates are also described in, for example, WO 2006/113683 or US20070014905.

The fermentation is carried out at a temperature which permits fermentation of the fermentable sugars. In general, the higher the temperature at which the fermentation can be carried out, the more economic the industrial process. Typically, the temperature at which the fermentation is carried out is from 25-42 deg Celsius. Suitable temperature ranges include 25-41, 26-40, 27-40, 28-40, 29-40, 30-40, 25-39, 26-39, 27-39, 28-39, 29-39, 30-39, 31-39, 32-39, 33-39, 25-38, 26-38, 27-38, 28-38, 29-38, 30-38, 31-38, 32-38, 33-38, 25-27, 26-37, 27-37, 28-37, 29-37, 30-37, 31-37, 32-37, 33-37, 25-36, 26-36, 27-36, 28-36, 29-36, 30-36, 31-36, 32-36, 33-36, 25-35, 26-35, 27-35, 28-35, 29-35, 30-35, 31-35, 32-35, 33-35 deg Celsius.

Methods for fermentation and distillation are known in the art and are described in, for example, WO 2006/113683 or US20070014905.

The inventors have further found that strain V09/024,011 is able to efficiently carry out fermentation of fermentable sugars following recycling of the yeast after fermentation. In this regard, the inventors have found that when strain V09/024,011 is recovered following fermentation of, for example, the fermentable sugars in sugar cane juice, the recovered strain can be used to efficiently ferment fresh fermentable substrate, such as sugar cane juice. The inventors have found that this Process of recycling strain V09/024,011 in the fermentation process can be repeated many times without greatly affecting fermentation efficiency.

Thus, the invention also relates to a method of producing ethanol comprising:
(a) incubating Saccharomyces strain V09/024,011 with a substrate comprising fermentable sugars under conditions which allow fermentation of the fermentable sugars to produce ethanol; and
(b) recovering strain V09/024,011 following fermentation of at least a portion of the fermentable sugars, and repeating step (a), and optionally step (b), with the recovered strain V09/024,011.

The invention further relates to a method of producing distiller's residue. As used herein, “distiller's residue” refers to the residue remaining after removal of ethanol following fermentation, or material produced from residue remaining after removal of ethanol following fermentation. The distiller's residue may be, for example, residual solids or liquids. In one form, the distiller's residue may be distiller's grains. Distiller's grains are typically solid residue remaining after removal of ethanol following fermentation, or material formed from such solid residue. The distiller's grains may be dry or wet distiller's grains. Distiller's grains may be produced from the residual solids produced in the fermentation using methods known in the art and described in, for example, U.S. Pat. No. 7,572,353. In other forms, the distiller's residue may be, for example, stillage, dunder or vinasse. Because Saccharomyces strain V09/024,011 reduces the level of residual sugars remaining following fermentation, the distiller's residue which results from fermentation using strain V09/024,011 has a lowered glucose content and is therefore more stable and less prone to charring, caramelisation or contamination with unwanted microorganisms.

The invention also provides a composition comprising a Saccharomyces cerevisiae strain having NMI accession no. V09/024,011. The composition may be, for example, cream yeast, compressed yeast, wet yeast, dry yeast, semi-dried yeast, crumble yeast, stabilized liquid yeast or frozen yeast. Methods for preparing such yeast compositions are known in the art.

### Test T1

Step 1: Yeast strains are streaked onto 2% w/v D-glucose 1% bacteriological peptone and 0.5% yeast extract medium solidified with 2% agar using standard microbiological techniques.

Step 2: After incubation for 72 hours at 30 deg Celsius, yeast cells are taken from plates using a sterile microbiological loop and inoculated to an OD₆₀₀ (Optical Density at 600 nm) of between 0.1 and 0.2 units (OD₆₀₀ at T₀) in 50 ml of broth containing xylose (5% w/v), Difco Yeast Nitrogen Base w/o amino acids (0.67%), citric acid (0.3%) and trisodium citrate (0.7%) in distilled water in a 250 ml Erlenmeyer flask. An OD₆₀₀ of 0.1 unit is equal to approximately 9×10⁵ yeast cells/mL. D-(+)-Xylose, minimum 99% can be obtained from Sigma-Aldrich.

Step 3: Cultures are incubated at 30 deg Celsius with shaking at 220 rpm (10 cm orbital diameter) for 48 hours.

Step 4: After 48 hours incubation, OD₆₀₀ of culture is measured (OD₆₀₀ at T₄₈).

Step 5: The fold increase in biomass is determined by the equation: OD₆₀₀ at T₄₈/OD₆₀₀ at T₀.

The invention will now be described in detail by way of reference only to the following non-limiting examples.

### EXAMPLES

### Example 1

### Isolation of Saccharomyces cerevisiae Strain V09/024,011

Saccharomyces cerevisiae strain V09/024,011 was isolated from a population of Saccharomyces cerevisiae produced using the method described in WO2005/121337. In addition to being capable of growth using xylose as a sole carbon source, strain V09/024,011 was able to efficiently produce ethanol from fermentable sugars.

Strain V09/024,011 was verified to be a Saccharomyces cerevisiae strain by its ability to sporulate and produce progeny when the germinated spores were mated with standard strains of Saccharomyces cerevisiae, including tester strains of Saccharomyces cerevisiae. One such tester strain is W303-1A. Specifically, germinated spores of strain V09/024,011 were able to produce progeny when mated with germinated spores of tester strain W303-1A.

In more detail, strain W303-1A (which was obtained from the Yeast Genetic Stock Center at the ATCC, USA (ATCC #208352) and strain V09/024,011 were cultured to form haploid Saccharomyces yeast as described in Ausubel, F. M. et al. (1997), Current Protocols in Molecular Biology, Volume 2, pages 13.2.1 to 13.2.5, published by John Wiley & Sons. Subsequently, the spores were germinated on a solid medium such as GYP containing 1% w/v D-glucose, 0.5% yeast extract, 1% w/v bacteriological peptone and 1.5% w/v agar and incubated at 30° C. for three to five days. The isolated germinated spores from strain V09/024,011 and W303-1A were then mated together using the method described in, for example, Ausubel, F. M. et al. (1997), Current Protocols in molecular Biology, Volume 2, pages 13.2.1 to 13.2.5, published by John Wiley & Sons.

Formation of hybrid zygotes could be observed under a microscope demonstrating that strain V09/024,011 is a Saccharomyces cerevisiae strain.

Saccharomyces cerevisiae strain V09/024,011 was deposited under the Budapest Treaty at the National Measurement Institute, 1/153 Bertie Street, Port Melbourne, Victoria, 3207, Australia, on 23 Sep. 2009 under accession number NMI V09/024,011.

Strains Fall, Thermosacc Dry and Ethanol Red and BG-1, which are representative of Saccharomyces yeast used for industrial ethanol production, were isolated using standard microbiological procedures from commercial packs of dry alcohol yeast. Ethanol Red was obtained from Fermentis, BP 3029-137 rue Gabriel Péri, F-59703 Marcq-en-Baroeul Cedex France. Fali was obtained from Fleishmann's Yeast, 1350 Timberlake Manor Parkway, Suite 550, Chesterfield, Mo. 63017, USA. Thermosacc Dry was obtained from Lallemand Ethanol Technology, 6120 W. Douglas Avenue, Milwaukee, Wis. 53218, USA. BG-1 was obtained from Copersucar, Brazil (L. C. Basso et al., 2008, FEMS Yeast Research, 8:1155-1163).

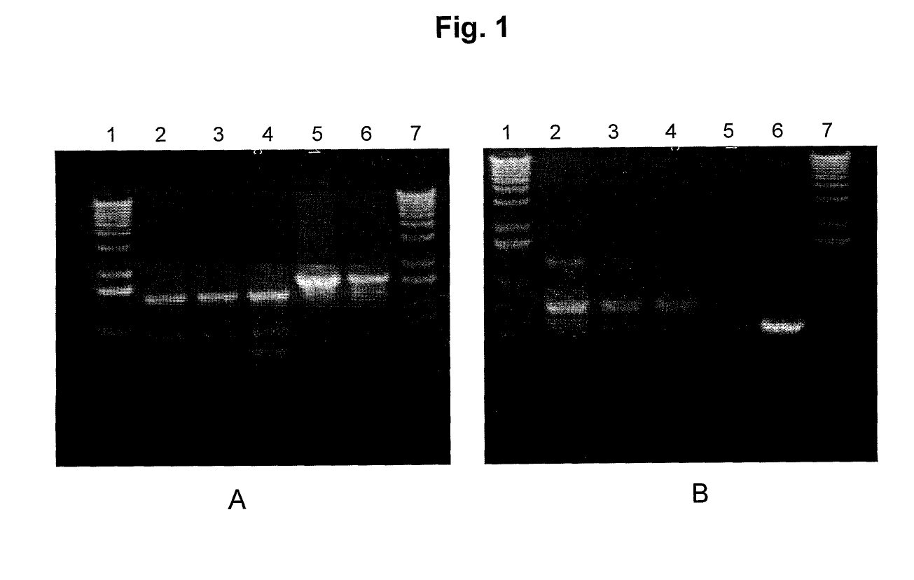

To determine if strain V09/024,011 was different from Fali, Thermosacc Dry, Ethanol Red and BG-1, a genetic fingerprint was performed by amplifying sequence located between adjacent Ty1 or Ty3 transposon elements in the yeast genome using PCR. In this regard, yeast chromosomal DNA was extracted using methods described in Bell P. J., Letters in Applied Microbiology 2004, 38: 388-392. To amplify the sequence located between transposon elements Ty1 and Ty3 primer regions, 5 microlitres of a 1:10 dilution of yeast chromosomal DNA was added to a PCR reaction mix containing 5 microlitres of 25 mM magnesium chloride, 5 microlitres of PCR buffer, 0.625 microlitre of 25 mM dNTP's, 1 microlitre of primer Ty1R1 (5′ CYTCTAACCTTCGATGACAGCTTCTC 3′) or Ty3R1 (5′ GGAAGGWCGGGTTTTGTCTCATGTTG 3′), 0.25 microlitre of Amplitaq Gold and 32.125 microlitres of sterile distilled water. The reaction mix was incubated at 95 deg Celsius for 15 min to denature the DNA and activate the Amplitaq Gold, then subjected to 35 cycles of 95 deg Celsius for 1 min, 50 deg Celsius for 1 min and 72 deg Celsius for 1 min and 45. Electrophoreis of PCR products used a gel of 1.5% w/v agarose in TAE buffer pH 8, which was 40 mM Tris acetate+1 mM EDTA, run for 20 min at 90 V and stained with ethidium bromide using standard conditions. The results are shown in FIGS. 1 A and B.

Referring to FIGS. 1 A and B, it can be seen that strain V09/024,011 produced a banding pattern that was distinct from Fali, Thermosacc Dry, Ethanol Red and BG-1. This indicates that Saccharomyces cerevisiae strain V09/024,011 is genetically distinct from Fali, Thermosacc Dry, Ethanol Red and BG-1. The results also demonstrate that the primers could not differentiate between Fali, Thermosacc Dry and Ethanol Red suggesting that these strains are very similar to each other, if not identical genetically.

### Example 2

### Growth of Strain V09/024,011 in Minimal Medium Containing Xylose as a Sole Carbon Source

Growth of strain V09/024,011 on xylose as a sole carbon source was determined using Test T1. Saccharomyces cerevisiae strain V09/024,011 was streaked onto 2% w/v D-glucose 1% bacteriological peptone and 0.5% yeast extract medium (GYP) solidified with 2% agar using standard microbiological techniques. After incubation for 72 hours at 30 deg Celsius, yeast cells were taken from plates using a sterile microbiological loop and inoculated to an OD₆₀₀ (Optical Density at 600 nm) of between 0.1 and 0.2 units (OD₆₀₀ at T0) in 50 ml of broth. An OD₆₀₀ of 0.1 unit is equal to approximately 9×10⁵ yeast cells/mL. The broth contained xylose (5% w/v), Difco Yeast Nitrogen Base w/o amino acids (0.67%), citric acid (0.3%) and trisodium citrate (0.7%) in distilled water in a 250 ml Erlenmeyer flask. Citric acid and trisodium citrate were provided as buffering agents that are not able to be used as growth substrates by Saccharomyces. D-(+)-Xylose 99% pure was obtained from Sigma-Aldrich (catalogue number X1500-500G). Cultures were incubated at 30 deg Celsius with shaking at 220 rpm (10 cm orbital diameter) for 48 hours prior to measuring OD₆₀₀ (OD₆₀₀ at T₄₈ hrs). The fold increase in biomass was determined by the equation: OD₆₀₀ at T₄₈ hrs divided by OD₆₀₀ at T₀.

Strain V09/024,011 was inoculated at an initial OD₆₀₀ of 0.11 and increased more than 4-fold in 48 hours. Under the same conditions biomasses of BG-1, Fali, Thermosacc Dry and Ethanol Red yeast strains increased less than two-fold.

### Example 3

### Alcoholic Fermentation of Corn Mash by Freshly Grown Yeast

Corn mash was prepared from corn (Zea maize) that was ground by passing three-times through a plate grinder, followed by grinding in a coffee grinder. Ninety-five percent of the ground corn had a particle size of equal to or less than 850 micrometers (less than 20 mesh). Ground corn was suspended in 1 mM CaCl₂ in deionized water to give 34% solids at 55 deg Celsius. Alpha-amylase (Liquozyme Supra, Novozymes) was added at a rate of 0.25 mL per 100 mL of suspended ground corn and temperature of the mixture raised to 97 deg Celsius. The mixture was continually stirred for 60 min. Temperature was reduced to 80 deg Celsius and a further 0.25 mL alpha-amylase added per 100 mL of mixture. The liquefied corn mash was stirred continually for a further 30 min. Temperature was brought to 34 deg Celsius and urea added to a final concentration of 16 mM. Maltodextrin was added to a final concentration of 16% w/v. Glucoamylase (Spirizyme, Novozymes) was mixed into the maltodextrin-fortified corn mash at the rate of 0.04 mL per 100 mL of corn mash. The corn mash was then dispensed in 150 mL amounts to 250 mL Erlenmeyer flasks.

Initial analysis of the ability of strains Fali, Thermosacc Dry and Ethanol Red to produce ethanol from corn mash at 34 deg Celsius indicated that each of these strains capabilities for producing ethanol are very similar. This is consistent with the PCR results of Example 1, which shows that Fali, Thermosacc Dry and Ethanol Red are either identical or very closely related. Ethanol Red was therefore chosen as a representative strain of Saccharomyces yeast used for industrial starch-to-ethanol production for comparison with strain V09/024,011.

Strains V09/024,011 and Ethanol Red were grown and harvested as described by Myers et al. (Applied and Environmental Microbiology, 1997, Vol. 63, pp. 145-150). After harvesting 0.33 g of yeast cells were suspended in 5 mL 2% w/v D-glucose, 1% bacteriological peptone and 0.5% yeast extract (GYP) at 34 deg Celsius for 30 min prior to inoculating 3.8 mL into the 150 mL fortified corn mash. Flasks were placed on an orbital shaker at 34 deg Celsius and 150 rpm.

Samples of 1 mL were taken at intervals and centrifuged at 13,000 rpm for 3 min in Eppendorf tubes. Supernatants were analyzed for glucose, glycerol, acetate and ethanol by HPLC using a Bio-Rad Laboratories Inc. Aminex HPX-87 H column (catalogue number 125-0140) with Cation-H Guard column (Catalogue number 125-0129). Mobile phase was 4 mM sulphuric acid in HPLC-grade water at a flow rate of 0.6 mL per min and a column temperature of 35 deg Celsius.

TABLE 1
Amounts of ethanol, acetate, glycerol and residual glucose in
corn mash fermentations inoculated with freshly grown yeasts
and following incubation at 34 deg Celsius for 72 hours.

| Strain | Glucose | Glycerol | Acetate | Ethanol |
|---|---|---|---|---|
| V09/024011 | 0.06 | 0.93 | 0.11 | 13.5 |
| Ethanol Red | 1.66 | 1.04 | 0.12 | 12.5 |

Values are % weight per volume.

These data show that under the conditions described, strain V09/024,011 utilized virtually all the available glucose released through amylase and glucoamylase digestion of starch and maltodextrin, whereas there was significant residual glucose present in the fermentation carried out using strain Ethanol Red. Moreover, relative to strain Ethanol Red, strain V09/024,011 converted more glucose into ethanol and produced less glycerol and acetate, which are unwanted by-products.

Characteristics exhibited by strain V09/024,011 provide several advantages for starch ethanol producers. These include ability to improve yield and productivity of ethanol from starchy materials by producing increased titres of ethanol, by producing lower amounts of by-products such as glycerol and acetate, having increased potential to recycle water in the process due to lowered accumulation of inhibitors such as acetate, higher protein content in distiller's residue due to lowered residual sugar content, and reduced distillation costs and waste water due to the reduced proportion of water relative to ethanol.

### Example 4

### Alcoholic Fermentation of Corn Mash by Freshly Grown Yeast Under Conditions with Excess Sugar

Corn mash was prepared from corn (Zea maize) that was ground by passing three-times through a plate grinder. Eighty-five percent of the ground corn had a particle size of equal to or less than 850 micrometers (less than 20 mesh). Ground corn was suspended in 1 mM CaCl₂ in de-ionized water to give 34% solids at 55 deg Celsius. Alpha-amylase (Liquozyme Supra, Novozymes) was added at a rate of 0.25 mL per 100 mL of suspended ground corn and temperature of the mixture raised to 97 deg Celsius. The mixture was continually stirred for 60 min. Temperature was reduced to 80 deg Celsius and a further 0.25 mL alpha-amylase added per 100 mL of mixture. The liquefied corn mash was stirred continually for a further 30 min. Temperature was brought to 34 deg Celsius and urea added to a final concentration of 16 mM. Maltodextrin was added to a final concentration of 16% w/v. Glucoamylase (Spirizyme, Novozymes) was mixed into the maltodextrin-fortified corn mash at the rate of 0.04 mL per 100 mL of corn mash. The corn mash was then dispensed in 150 mL amounts to 250 mL Erlenmeyer flasks.

Strain Ethanol Red, which is a representative of Saccharomyces yeast used for industrial starch-to-ethanol production, was isolated using standard microbiological procedures from a pack of “Ethanol Red” dry alcohol yeast which is commercially available from Fermentis, BP 3029-137 rue Gabriel Péri, F-59703 Marcq-en-Baroeul Cedex France (batch number 470/2, production date 10/2006). Strains V09/024,011 and Ethanol Red were grown and harvested as described by Myers et al. (Applied and Environmental Microbiology, 1997, Vol. 63, pp. 145-150). After harvesting 0.33 g of yeast cells were suspended in 5 mL 2% w/v D-glucose, 1% bacteriological peptone and 0.5% yeast extract (GYP) at 34 deg Celsius for 30 min prior to inoculating 3.8 mL into the 150 mL fortified corn mash. Flasks were placed on an orbital shaker at 34 deg Celsius and 150 rpm.

Samples of 1 mL were taken at intervals and centrifuged at 13,000 rpm for 3 min in Eppendorf tubes. Supernatants were analyzed for glucose, glycerol, acetate and ethanol by HPLC using a Bio-Rad Laboratories Inc. Aminex HPX-87 H column (catalogue number 125-0140) with Cation-H Guard column (Catalogue number 125-0129). Mobile phase was 4 mM sulphuric acid in HPLC-grade water at a flow rate of 0.6 mL per min and a column temperature of 35 deg Celsius.

TABLE 2
Amounts of ethanol, acetate, glycerol and residual glucose in
corn mash fermentations inoculated with freshly grown yeasts
and following incubation at 34 deg Celsius for 72 hours.

| Strain | Glucose | Glycerol | Acetate | Ethanol |
|---|---|---|---|---|
| V09/024011 | 5.1 | 1.0 | 0.12 | 13.5 |
| Ethanol Red | 6.5 | 1.1 | 0.14 | 12.8 |

Values are % weight per volume.

These data show that under the conditions described in this test, there was a significant quantity of sugar remaining in both strain V09/024,011 and strain Ethanol Red. However, comparison between the two strains indicates that strain V09/024,011 fermented more of the sugar, and this is reflected in the higher ethanol concentration achieved. As in Example 3, relative to strain Ethanol Red, strain V09/024,011 converted more glucose into ethanol and produced less glycerol and acetate, which are unwanted by-products.

Characteristics exhibited by strain V09/024,011 provide several advantages for starch ethanol producers. These include ability to improve yield and productivity of ethanol from starchy materials by producing increased titres of ethanol, by producing lower amounts of by-products such as glycerol and acetate, having increased potential to recycle water in the process due to lowered accumulation of inhibitors such as acetate, higher protein content in distiller's residue due to lowered residual sugar content, and reduced distillation costs and waste water due to the reduced proportion of water relative to ethanol.

### Example 5

### Alcoholic Fermentation of Corn Mash by Active Dried Yeast Under Low Sugar Conditions

Yeast strain V09/024,011 was grown and harvested according to Myers et al. (Applied and Environmental Microbiology, 1997, Vol. 63, pp. 145-150) except that 30 mM NaCl was added to the culture and the temperature raised to 37 deg Celsius in the last 3 hours of propagation. Yeast was dried using methods well known to those skilled in the art and described for example in U.S. Pat. Nos. 4,370,420 and 6,372,481. Dry yeast was rehydrated by gently sprinkling 0.1 g dry yeast on top of 5 mL GYP prewarmed to 37 deg Celsius and leaving static for 30 min. After the 30 min incubation period, yeast was mixed by gentle stirring prior and 3.8 mL then inoculated into 150 mL maltodextrin-fortified corn mash, which was prepared as described in Example 3. Dried Ethanol Red yeast, commercially available from Fermentis, BP 3029-137 rue Gabriel Péri, F-59703 Marcq-en-Baroeul Cedex France, was rehydrated and inoculated under identical conditions.

TABLE 3
Amounts of ethanol, acetate, glycerol and residual glucose in
corn mash fermentations inoculated with rehydrated dry yeasts
and following incubation at 34 deg Celsius for 72 hours.

| Strain | Glucose | Glycerol | Acetate | Ethanol |
|---|---|---|---|---|
| V09/024011 | 0.02 | 0.89 | 0.11 | 12.2 |
| Ethanol Red | 0.32 | 0.97 | 0.13 | 11.9 |

Values are % weight per volume.

These data show that under the conditions described in this test, there was a low concentration of sugar remaining with both strain V09/024,011 and strain Ethanol Red. However, comparison between the two sugar concentrations indicates that the strain V09/024,011 fermented more of the sugar, and this is reflected in the higher ethanol concentration achieved. As in Examples 3 and 4, relative to strain Ethanol Red, strain V09/024,011 converted more glucose into ethanol and produced less glycerol and acetate, which are unwanted by-products.

Characteristics exhibited by strain V09/024,011 provide several advantages for starch ethanol producers. These include ability to improve yield and productivity of ethanol from starchy materials by producing increased titres of ethanol, by producing lower amounts of by-products such as glycerol and acetate, having increased potential to recycle water in the process due to lowered accumulation of inhibitors such as acetate, higher protein content in distiller's residue due to lowered residual sugar content, and reduced distillation costs and waste water due to the reduced proportion of water relative to ethanol.

Examination of the data obtained in Examples 3, 4 and 5 reveals strain V09/024,011 is consistently capable of producing higher levels of ethanol from corn mash under a variety of conditions. In addition, in each case, the residual sugars and the quantity of by-products produced during the fermentation by V09/024,011 was lower than that observed with strain Ethanol Red.

### Example 6

### Alcoholic Fermentation of Sugar Cane Juice by Yeast Following Multiple Recyclings

The Brazilian sugar cane juice fermentation process involves intensive recycling of inoculated yeast whereby >90% of the yeast is reused from one fermentation to the next. However, high ethanol concentrations, high temperature, osmotic stress due to sugar and salts, acidity, and competing organisms such as bacteria and “wild” yeasts are recognized stresses faced by the inoculated yeast and these stresses impact negatively on fermentation yields and rates. Thus, as more recycling of the yeast occurs fermentation times become longer and residual sugar left after fermentation increases.

Yeast strain BG-1 represents one of the most widely used yeast strains in Brazilian ethanol plants (L. C. Basso et al., 2008, FEMS Yeast Research, 8:1155-1163). Yeast strain BG-1 and yeast strain V09/024,011 were grown as described by Myers et al. (Applied and Environmental Microbiology, 1997, Vol. 63, pp. 145-150). They were inoculated separately into 150 mL clarified sugar cane juice contained in cotton wool plugged 500 mL conical flasks at a density of 3×10e9 cells per mL and incubated at 34 degrees Celsius and 100 rpm for 20 hours. Cells were then harvested by centrifugation at 3,000×g and room temperature before being re-inoculated into sugar cane juice and incubated under identical conditions. This recycling procedure was repeated 10 times.

TABLE 4
Amounts of ethanol, glycerol, acetate and
residual sugars in cane juice fermentations inoculated
with recycled yeasts following incubation at 34 deg
Celsius for 20 hours.

| Strain | Sucrose | Glucose | Fructose | Glycerol | Acetate | Ethanol |
|---|---|---|---|---|---|---|
| V09/ | 0.0 | 0.0 | 0.0 | 0.59 | 0.05 | 9.6 |
| 024011 | | | | | | |
| BG-1 | 0.05 | 0.42 | 2.62 | 0.76 | 0.09 | 7.9 |

Values are % weight per volume

These data show that after 10 recyclings, strain V09/024,011 remained vigorously fermentative, utilising all available sucrose, glucose and fructose. By comparison the industrially prevalent yeast strain BG-1, showed less fermentative activity leaving residual sucrose, glucose and fructose within the same fermentation period. Strain V09/024,011 also accumulated less glycerol and acetate (unwanted byproducts) than strain BG-1. These data indicate that strain V09/024,011 is more capable of withstanding the stresses associated with recycling through sugar cane juice fermentations than the industrially prevalent strain BG-1.

### Example 7

### Alcoholic Fermentation of Sorghum Mash by Active Dried Yeast

Ground sorghum was suspended in 1 mM CaCl₂ in deionized water to give 36% solids at 55 deg Celsius. Alpha-amylase (Liquozyme Supra, Novozymes) was added at a rate of 0.25 mL per 100 mL of suspended ground sorghum and temperature of the mixture raised to 97 deg Celsius. The mixture was continually stirred for 60 min. Temperature was reduced to 80 deg Celsius and a further 0.25 mL alpha-amylase added per 100 mL of mixture. The liquefied sorghum mash was stirred continually for a further 30 min. Temperature was brought to 34 deg Celsius and urea added to a final concentration of 16 mM. Glucoamylase (Spirizyme, Novozymes) was mixed into the sorghum mash at the rate of 0.04 mL per 100 mL of mash. The mash was then dispensed in 150 mL amounts to 250 mL Erlenmeyer flasks.

Yeast strain V09/024,011 was grown and harvested according to Myers et al. (Applied and Environmental Microbiology, 1997, Vol. 63, pp. 145-150) except that 30 mM NaCl was added to the culture and the temperature raised to 37 deg Celsius in the last 3 hours of propagation. Yeast was dried using methods well known to those skilled in the art and described for example in U.S. Pat. Nos. 4,370,420 and 6,372,481. Dry yeast was rehydrated by gently sprinkling 0.1 g dry yeast on top of 5 mL GYP prewarmed to 37 deg Celsius and leaving static for 30 min. After the 30 min incubation period, yeast was mixed by gentle stirring prior and 3.8 mL then inoculated into 150 mL sorghum mash, which was prepared as described in Example 3. Fali Ethanol Dry Yeast, commercially available from AB Mauri Fleishchmann's Yeast, 1350 Timberlake Manor Parkway, Suite 550, Chesterfield, Mo. 63017, U.S.A. was rehydrated and inoculated under identical conditions.

TABLE 5
Amounts of ethanol, glycerol and residual glucose in sorghum
mash fermentations inoculated with rehydrated dry yeasts and
following incubation at 34 deg Celsius for 48 hours.

| Strain | Glucose | Glycerol | Ethanol |
|---|---|---|---|
| V09/024011 | 1.8 | 0.52 | 10.5 |
| Fall yeast | 2.5 | 0.84 | 9.9 |

Values are % weight per volume.

These data show that under the conditions described in this test, the strain V09/024,011 fermented more of the sugar, and this is reflected in the higher ethanol concentration achieved relative to control strain Fali yeast. Strain V09/024,011 also produced less glycerol, which is an unwanted by-product, than Fall yeast.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. An isolated Saccharomyces cerevisiae strain having NMI accession no. V09/024,011.

2. A method of producing ethanol, comprising incubating a Saccharomyces cerevisiae strain having NMI accession no. V09/024,011 with a substrate comprising fermentable sugars under conditions which allow fermentation of the fermentable sugars to produce ethanol.

3. The method of claim 2, comprising isolating the ethanol.

4. The method of claim 2, wherein the fermentable sugar in the substrate comprises hydrolysed starch, hydrolysed cellulose, hydrolysed maltodextrin, syrup, sugar cane juice, molasses, sugar beet juice, grape juice, or fruit juice.

5. The method of claim 4, wherein the fermentable sugar is hydrolysed starch and the hydrolysed starch is obtained from one or more of the crops corn, wheat, barley, cassava, sorghum, sweet potato, millet, and rice.

6. Ethanol produced by the method of claim 2.

7. (canceled)

8. A method of producing distiller's residue comprising incubating a Saccharomyces cerevisiae strain having NMI accession no. V09/024011 with a substrate comprising fermentable sugar under conditions which allow fermentation of the fermentable sugar to produce ethanol and distiller's residue.

9. The method of claim 8, comprising isolating the distiller's residue.

10. (canceled)

11. (canceled)

12. The method of claim 8, wherein the fermentable sugar comprises hydrolysed starch, hydrolysed cellulose, hydrolysed maltodextrin, syrup, sugar cane juice, molasses, sugar beet juice, grape juice, or fruit juice.

13. The method of claim 12, wherein the source of fermentable sugar is hydrolysed starch and the hydrolysed starch is obtained from one or more of the crops corn, wheat, barley, cassava, sorghum, sweet potato, millet, and rice.

14. Distiller's residue produced by the method of claim 8.

15. A method of producing ethanol comprising:
(a) incubating Saccharomyces cerevisiae strain V09/024,011 with a substrate comprising fermentable sugars under conditions which allow fermentation of the fermentable sugars to produce ethanol; and
(b) recovering Saccharomyces cerevisiae strain V09/024011 following fermentation of at least a portion of the fermentable sugars, and repeating step (a), and optionally step (b), with the recovered Saccharomyces cerevisiae strain V09/024,011.

16. The method of claim 15, wherein the substrate comprises sugar cane juice.

17. A composition comprising Saccharomyces cerevisiae strain V09/02411.
